(19) United States
(12) Patent Application Publication
(10) Pub. No.: US 2022/0280682 A1
(43) Pub. Date: Sep. 8, 2022

(54) ANTIMICROBIAL COMPOSITIONS AND ARTICLES COMPRISING THE SAME

(71) Applicant: 3M INNOVATIVE PROPERTIES COMPANY, St. Paul, MN (US)

(72) Inventors: Rajan B. BODKHE, Woodbury, MN (US); Naimul KARIM, Maplewood, MN (US); Matthew T. SCHOLZ, Woodbury, MN (US)

(21) Appl. No.: 17/630,032

(22) PCT Filed: Jul. 13, 2020
(86) PCT No.: PCT/IB2020/056553

§ 371 Date: Jan. 25, 2022

Related U.S. Application Data
(60) Provisional application No. 62/879,752, filed on Jul. 29, 2019.

(51) Int. Cl. A61L 15/46; A61L 15/22; A61L 29/16; A61L 29/08

(52) U.S. Cl. CPC A61L 15/46; A61L 15/22; A61L 29/16; A61L 29/085; A01N 25/34

(57) ABSTRACT

The present disclosure provides a solid composition that includes a polycarboxylic acid chelator component mixed with a water-soluble plasticizer component, and a water-soluble or water-dispersible polymer dissolved and/or dispersed in the plasticizer component. The water-soluble plasticizer component has a boiling point >105° C. and has a formula weight of less than 5000 atomic mass units. The composition comprises <10 wt % of a solvent that has a boiling point ≤100 degrees C. When mixed with deionized water at a 1:9 mass ratio, the composition forms an aqueous mixture having a pH of about 2.5-5.5. Articles that include a substrate with a first major surface having a layer comprising the composition adhered to the first major surface are also provided. Methods of treating a biofilm or a wound site with the articles are also provided.

### TECHNICAL FIELD

The present disclosure relates to articles and antimicrobial compositions containing a chelator compound comprising a polycarboxylic acid compound, a water-soluble or water-dispersible polymer, and a water-soluble plasticizer component.

### BACKGROUND

Microbes are found virtually everywhere, often in high concentrations, and are responsible for a significant amount of disease and infection. Killing and/or eliminating these microorganisms is desirable for a variety of reasons.

Bacteria present special challenges because they can exist in a number of forms (e.g., planktonic, spore and biofilm) and their self-preservation mechanisms make them extremely difficult to treat and/or eradicate. For example, the bacteria in biofilms or spores are down-regulated (sessile) and not actively dividing, which makes them resistant to attack by a large group of antibiotics and antimicrobials that attack the bacteria during the active parts of their lifecycle, e.g., cell division.

In a biofilm, bacteria interact with and adhere to surfaces and form colonies which facilitate continued growth. The bacteria produce exopolysaccharide (EPS) and/or extra cellular-polysaccharide (ECPS) macromolecules that keep them attached to the surface and form a protective barrier effective against many forms of attack. Protection most likely can be attributed to the small diameter of the flow channels in the matrix, which restricts the size of molecules that can reach the underlying bacteria, and consumption of biocides through interactions with portions of the EPS/ECPS macromolecular matrix and bacterial secretions and waste products contained therein. (Certain fungi also can form biofilms, many of which present the same types of challenges presented here.)

Bacteria also can form spores, which are protein/polysaccharide shells or coatings having reduced permeability and susceptibility. Spores provide additional resistance to eradication efforts by preventing attack from materials that are harmful to the bacteria.

Due to the protection afforded by a macromolecular matrix (biofilm) or shell (spore) and their down-regulated state, bacteria in these states are very difficult to treat. The types of biocides and antimicrobials effective in treating bacteria in this form are gases (e.g. steam, ethylene oxide, etc.) as well as strongly acidic and/or oxidizing compositions, often involving halogen atoms, oxygen atoms, or both. Common examples include hypochlorite Solutions (e.g., bleach), phenolics, mineral acids (e.g., HCl), H₂O₂, and the like. Large dosages of Such chemicals must be allowed to contact the biofilm or spore for extended amounts of time to be effective, which makes them impractical for many applications.

Animal tissue wounds present both a good environment for bacterial, and even biofilm, growth and a Surface or Substrate requiring gentle treatment, thus making a difficult problem even worse.

Nosocomial or hospital acquired infections (HAIs) can be caused by viral, bacterial, and/or fungal pathogens and can involve any system of the body. HAIs are a leading cause of patient deaths, and they increase the length of hospitalizations for patients, mortality and healthcare costs; in the developed world, they are estimated to occur in 5-10% of all hospitalizations, even higher for pediatric and neonatal patients. They often are associated with medical devices or blood product transfusions. Three major sites of HAIs are bloodstream, respiratory tract, and urinary tract. Most patients who have HAIs have invasive supportive measures such as central intravenous lines, mechanical ventilation, and catheters, which provide an ingress point for pathogenic organisms. Ventilator-associated pneumonia can be caused by Staphylococcus aureus, methicillin-resistant Staphylococcus aureus (MRSA), Candida albicans, Pseudomonas aeruginosa, Acinetobacter baumannii, Stenotrophomonas maltophilia, Clostridium difficile, and Mycobacterium tuberculosis, while other HAIs include urinary tract infections, pneumonia, gastroenteritis, Vancomycin-resistant Enterococcus (VRE), and Legionellosis.

Medical equipment such as endoscopes, gastroscopes, the flow-channels of hematology and dialyzer equipment, the airflow path of respiratory equipment, ISE, HPLC, and certain catheters are designed to be used multiple times. Significant risks have been associated with inadequate or improper cleaning due to the presence of residual soil and/or improper disinfection or sterilization, up to and including HAIs from contaminated devices such as bronchoscopes contaminated with Mycobacterium tuberculosis and the transmission of Hepatitis C virus to patients during colonoscopy procedures.

Bacteria also colonize both acute and chronic wounds and may exist in spore, planktonic, or biofilm forms. Often the bacterial contamination involves multiple species. Eradicating bacteria from wounds without retarding wound healing can be particularly challenging.

There remains a need for composition and articles that can be used in the treatment of microbes such as bacteria. Moreover, methods and articles capable of treating bacteria that colonize acute wounds at the time of injury and during all stages of healing, as well as in the treatment of chronic wounds, also are highly desirable.

### SUMMARY

The disclosed compositions and articles that can be used in treatment or elimination of microbes including but not limited to bacteria, regardless of whether they are in planktonic, or biofilm form and whether they are present as a single species or mixed culture.

The composition and articles comprise large proportions of polycarboxylic acid-comprising chelating components and, yet, remain surprisingly nonfriable and even flexible.

The compositions, when contacted with an aqueous environment (e.g., a wound site comprising blood, serum, or wound exudate) releases solutes that are lethal toward a wide spectrum of gram positive and Gram negative bacteria other microbes such as viruses, fungi, molds, and yeasts, that may be present in the environment.

Articles and methods for treating wound areas also are provided. Articles comprising (e.g., as a layer) the solid compositions can be applied to a wound area and can be left in place for a period of time effective to inhibit increase of and/or reduce numbers of microorganisms in the wound site. In certain embodiments, the article can be applied to a wound treatment area temporarily, allowed to release solutes into the wound environment for a period of time, and removed.

Further, HAIs can be prevented or remedied by applying the anti-microbial composition to a surface located in a medical treatment facility or to the surface of a medical device so as to prevent or remove a biofilm and/or kill bacteria adhered thereto. A patient possessing a HAI also can be treated with an antimicrobial composition or an article including or based thereon.

Additionally, the surfaces of permanently or removably implantable objects can be treated so as to prevent biofilm formation or, after implantation, can be treated to remove biofilm on such surfaces.

In one aspect, the present disclosure provides a first article. The first article can comprise a substrate having a first major surface and, optionally, a second major surface opposite the first major surface; and at least one layer adhered to the first major surface and, optionally, a second major surface opposite the first major surface; and at least one layer adhered to the first major surface, wherein the at least one layer comprises a composition. The composition can comprise a dicarboxylic acid or tricarboxylic acid chelator component, or a salt thereof, mixed with a water-soluble plasticizer component; and a water-soluble or water-dispersible polymer having a T_{G} greater than or equal to 20° C. dissolved and/or dispersed in the plasticizer component. The composition comprises at least about 10% (w/w) of the chelator component. The water-soluble plasticizer component has a boiling point greater than 105° C. and has a formula weight of less than 5000 atomic mass units. The composition comprises less than 10 wt % of a solvent that has a boiling point less than or equal to 100 degrees C. The composition is a solid at 25° C. The composition, when mixed with deionized water at a 1:9 mass ratio, forms an aqueous mixture having a pH of about 2.5-5.5.

In another aspect, the present disclosure provides a second article. The second article can comprise a substrate having a first major surface and, optionally, a second major surface opposite the first major surface; and at least one layer adhered to the first major surface and, optionally, a second major surface opposite the first major surface; and at least one layer adhered to the first major surface, wherein the at least one layer comprises a composition. The composition can comprise a tetracarboxylic acid chelator component, or a salt thereof, mixed with a water-soluble plasticizer component; and a water-soluble or water-dispersible polymer having a T_{G} greater than or equal to 20° C. dissolved and/or dispersed in the plasticizer component. The composition comprises at least about 10% (w/w) of the chelator component. The water-soluble plasticizer component has a boiling point greater than 105° C. and has a formula weight of less than 5000 atomic mass units. The composition comprises less than 10 wt % of a solvent that has a boiling point less than or equal to 100 degrees C. The composition is a solid at 25° C. The composition, when mixed with deionized water at a 1:9 mass ratio, forms an aqueous mixture having a pH of about 2.5-5.5.

In any of the above embodiments of the first or second article, the chelator compound comprises an aliphatic polycarboxylic acid or a salt thereof, an aromatic polycarboxylic acid or a salt thereof, or a combination thereof. In any of the above embodiments, the at least one layer can be flexible as defined by the ability to fold a coated film sample 180 degrees, creasing the fold by pinching between a thumb and first finger, unfolding the construction, removing one liner and observing that the film has not cracked or flaked; wherein the coated film sample comprises the at least one layer having a thickness of 150 microns and being disposed between two cured silicone release liners, each liner having a thickness of 50 microns. In any of the above embodiments, the chelator compound can be present in the composition up to about 60 wt %. In any of the above embodiments, the plasticizer component can be present in the composition at about 15 wt % to about 75 wt %. In any of the above embodiments, the water-soluble or water-dispersible polymer can be present in the composition at about 5 wt % to about 75wt %. In any of the above embodiments, the composition can be substantially water-free. In any of the above embodiments, the at least one layer can be about 50 microns thick to about 5000 microns thick. In any of the above embodiments, the article further can comprise a backing layer comprising a first side and a second side, wherein the substrate is adhered to the first side of the backing layer.

In yet another aspect, the present disclosure provides a first method of treating or preventing formation of a biofilm. The first method can comprise contacting a tissue with the at least one layer of the article of any one of the above embodiments of the first or second articles.

In yet another aspect, the present disclosure provides a method of treating a tissue to reduce a number of microorganisms residing therein or thereon. The method can comprise contacting the tissue with the at least one layer of the article of any one of the above embodiments of the first or second articles.

In any of the above embodiments of the first or second methods, contacting the tissue with the composition or article comprises contacting the tissue with the at least one layer of the article for about 2 hours to about 72 hours.

In yet another embodiment, the present disclosure provides a first composition. The first composition can comprise a dicarboxylic acid or tricarboxylic acid chelator component, or a salt thereof, mixed with a water-soluble plasticizer component; and a water-soluble or water-dispersible polymer having a T_{G} greater than or equal to 20° C. dissolved and/or dispersed in the plasticizer component. The first composition can comprise at least about 10% (w/w) of the chelator component. The water-soluble plasticizer component has a boiling point greater than 105° C. and has a formula weight of less than 5000 atomic mass units. The first composition comprises less than 10 wt % of a solvent that has a boiling point less than or equal to 100 degrees C. The first composition is a solid at 25° C. The first composition, when mixed with deionized water at a 1:9 mass ratio, forms an aqueous mixture having a pH of about 2.5-5.5.

In yet another embodiment, the present disclosure provides a second composition. The second composition can comprise a tetracarboxylic acid chelator component, or a salt thereof, mixed with a water-soluble plasticizer component; and a water-soluble or water-dispersible polymer having a T_{G} greater than or equal to 20° C. dissolved and/or dispersed in the plasticizer component. The second composition can comprise at least about 10% (w/w) of the chelator component. The water-soluble plasticizer component has a boiling point greater than 105° C. and has a formula weight of less than 5000 atomic mass units. The second composition comprises less than 10 wt % of a solvent that has a boiling point less than or equal to 100 degrees C. The second composition is a solid at 25° C. The second composition, when mixed with deionized water at a 1:9 mass ratio, forms an aqueous mixture having a pH of about 2.5-5.5.

“Microorganism” or “microbe” or “microorganism” refers to bacteria, yeast, mold, fungi, protozoa, mycoplasma, as well as viruses (including lipid enveloped RNA and DNA viruses).

“Antibiotic” means an organic chemical produced by microorganisms that has the ability in dilute concentrations to destroy or inhibit microorganisms and is used to treat infectious disease. This may also encompass semi-synthetic compounds that are chemical derivatives of the compound produced by microorganisms or synthetic compounds that act on very specific biochemical pathways necessary for the cell's survival.

“Antiseptic” means an antimicrobial component chemical agent that kills pathogenic and non-pathogenic microorganisms. Antiseptics generally interfere more broadly with the cellular metabolism and/or the cell envelope. Antiseptics are sometimes referred to as disinfectants, especially when used to treat hard surfaces. Suitable antiseptics include, for example: antimicrobial lipids; phenolic antiseptics; cationic antiseptics; iodine and/or iodophors; peroxide antiseptics; antimicrobial natural oils; or combinations thereof. These are described in US20180207122 incorporated herein by reference.

“Effective amount” means the amount of a chelator component and/or additional antimicrobial component when in a composition, as a whole, provides an antimicrobial (including, for example, antiviral, antibacterial, or antifungal) activity that reduces, prevents, or eliminates one or more species of microbes such that an acceptable level of the microbe results. Typically, this is a level low enough not to cause clinical symptoms and is desirably a non-detectable level. It should be understood that in the compositions, the concentrations or amounts of the components, when considered separately, may not kill to an acceptable level, or may not kill as broad a spectrum of undesired microorganisms, or may not kill as fast; however, when used together such components provide an enhanced (preferably synergistic) antimicrobial activity (as compared to the same components used alone under the same conditions).

“Treat” or “treatment” means to improve the condition of a subject relative to the affliction, typically in terms of clinical symptoms of the condition.

“Decolonization” refers to a reduction in the number of microorganisms (e.g., bacteria and fungi) present in or on tissue that do not necessarily cause immediate clinical symptoms. Examples of decolonization include, but are not limited to, decolonization of the nasal cavity and wounds. Ordinarily fewer microorganisms are present in colonized tissue than in infected tissue. When the tissue is completely decolonized the microorganisms have been “eradicated.”

“Subject” and “patient” includes humans, sheep, horses, cattle, pigs, dogs, cats, rats, mice, or other mammal.

“Affliction” means a condition to a body resulting from sickness, disease, injury, bacterial colonization, etc.

“Wound” refers to an injury to a subject which involves a break in the normal skin barrier exposing tissue below, which is caused by, for example, lacerations, surgery, burns, damage to underlying tissue such as pressure sores, poor circulation, and the like. Wounds are understood to include both acute and chronic wounds.

The terms “comprises”, and variations thereof do not have a limiting meaning where these terms appear in the description and claims.

As used herein, “a,” “an,” “the,” “at least one,” and “one or more” are used interchangeably. The term “and/or” means one or all of the listed elements (e.g., preventing and/or treating an affliction means preventing, treating, or both treating and preventing further afflictions).

Also herein, the recitations of numerical ranges by endpoints include all numbers subsumed within that range (e.g., 1 to 5 includes 1, 1.5, 2, 2.75, 3, 3.80, 4, 5, etc.).

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTIONS OF THE DRAWINGS

FIG. 1A is a plan view of one embodiment of an article according to the present disclosure.

FIG. 1B is an exploded side view of the article of FIG. 1A.

FIG. 2A is an exploded perspective view of an alternative embodiment of an article according to the present disclosure.

FIG. 2B is an exploded side view of the article of FIG. 2A.

### DETAILED DESCRIPTION

In one aspect, the present disclosure provides an antimicrobial composition that can be used in the treatment, elimination, and/or prevention of colonization of microbes in a wound site and/or on a medical device. Compositions of the present disclosure can be used to treat or prevent colonization of a wound site or a medical device with one or more infectious microorganisms. In particular, the compositions can be used to treat, or prevent formation of, a biofilm in a wound site and/or on a medical device.

Any surface that is or becomes moist is subject to biofilm formation. Thus, medical devices (e.g., catheters, stents, artificial joints, dental implants) intended for permanent or temporary placement on or into a patient. Extreme measures are taken to prevent biofilm formation because, once established, they are essentially impossible to eradicate in vivo and can cause life-altering, even lethal, infections.

The compositions can be used to provide effective topical antimicrobial activity and thereby treat and/or prevent a wide variety of afflictions. For example, they can be used in the treatment and/or prevention of afflictions that are caused, or aggravated by, microorganisms (e.g., Gram positive bacteria, Gram negative bacteria, fungi, protozoa, mycoplasma, yeast, viruses, and even lipid-enveloped viruses) on skin and/or mucous membranes, such as those in the nose (anterior nares, nasopharangyl cavity, nasal cavities, etc.), outer ear, and middle ear, mouth, rectum, vagina, or other similar tissues. Particularly relevant organisms that cause or aggravate such afflictions include Staphylococcus spp., Streptococcus spp., Pseudomonas spp., Enterococcus spp., and Esherichia spp., bacteria, as well as herpes virus, Aspergillus spp., Fusarium spp. Candida spp. as well as combinations thereof. Particularly virulent organisms include Staphylococcus aureus (including resistant strains such as Methicillin Resistant Staphylococcus aureus (MRSA), Staphylococcus epidermidis, Streptococcus pneumoniae, Enterococcus faecalis, Vancomycin Resistant Enterococcus (VRE), Pseudomonas aeruginosa, Escherichia coli, Aspergillus niger, Aspergillus fumigatus, Aspergillus clavatus, Fusarium solani, Fusarium oxysporum, Fusarium chlamydosporum, Candida albicans, Candida glabrata, Candida krusei, and combinations thereof.

Compositions can be used on a wide variety of surfaces. For example, they can be used on mammalian tissues (particularly, skin, mucosal tissue, chronic wounds, acute wounds, burns, and the like) and hard surfaces such as medical (e.g., surgical) devices, floor tiles, countertops, tubs, dishes, as well as on gloves (e.g., surgical gloves). They can also be delivered from swabs, cloth, sponges, foams, nonwovens, and paper products (e.g., paper towels and wipes), for example.

It should be understood that the compositions can be used in situations in which there are no clinical indications of an affliction. For example, the compositions can be used in methods of decolonizing at least a portion of an acute or chronic wound or other tissue surface that is colonized with bacteria, e.g. the nasal cavities (i.e., space behind the vestibule of the nose), anterior nares (i.e., the opening in the nose to the nasal cavities, also referred to as the external nares), and/or nasopharynx (i.e., the portion of the pharynx, i.e., throat, that lies above the point of food entry into the pharynx), esophagus, vaginal cavity etc.

Decolonization methods using the compositions may be particularly useful in immunocompromised patients (including oncology patients, diabetics, HIV patients, transplant patients and the like), particularly for fungi such as Aspergillus spp. and Fusarium spp.

In particular, the compositions can be used in chronic wounds to eliminate methicillin-resistant Staphylococcus aureus, which may or may not show clinical signs of infection such as inflammation, pus, exudate, etc.

In some embodiments, the compositions are substantive for relatively long periods of time to ensure adequate efficacy. For example, certain compositions remain at the site of application with antimicrobial activity for at least 4 hours and more preferably at least 8 hours, more preferably at least 24 hrs, and even more preferably at least 48 hours.

In some embodiment, the compositions are physically stable. As defined herein “physically stable” compositions are those that do not significantly change due to substantial precipitation, crystallization, phase separation, and the like, from their original condition during storage at 23.degree. C. for at least 3 months, and preferably for at least 6 months.

Solid compositions of the present disclosure, when disposed as a free thin film (i.e., not coated on a substrate) (or coating (e.g., approximately 0.05 mm thick to approximately 5 mm thick) on a substrate, are flexible and can be deformed without breaking, shattering, or flaking of the film or coating.

Compositions of the present disclosure comprise an effective amount of a polycarboxylic acid chelator compound. The amount is effective to prevent growth of a microorganism and/or to kill microorganisms on a surface to which the composition is contacted. A mucosal model to study microbial biofilm development and anti-biofilm therapeutics, Journal of Microbiological Methods 92 (2013) 201-208.

In certain embodiments, the polycarboxylic acid chelator compound, whether aliphatic, aromatic, or a combination thereof, comprises at least two carboxylic acid groups. In certain embodiments, the polycarboxylic acid chelator compound, whether aliphatic, aromatic or a combination thereof, comprises at least three carboxylic acid groups. In certain embodiments, the polycarboxylic acid chelator compound, whether aliphatic or aromatic, comprises at least four carboxylic acid groups.

Polycarboxylic acid- containing chelator compounds suitable for use in the inventive compositions include aliphatic polycarboxylic acids, aromatic polycarboxylic acids, compounds with both one or more aliphatic carboxylic acids and one or more aromatic carboxylic acids and salts or mixtures thereof. Nonlimiting examples of suitable polycarboxylic acid-containing chelator compounds include citric acid, glutaric acid, glutamic acid, maleic acid, succinic acid, tartaric acid, malic acid, ethylenediaminetetraacetic acid, phthalic acid, trimesic acid, and pyromellitic acid.

Preferred salts include those formed from monovalent inorganic bases and include cations such as K+, Na+, Li, and Ag+ and mixtures thereof. In some compositions polyvalent bases may be appropriate and include cations such as Ca++, Mg++, Zn++, Alternatively, the salt of the polycarboxylic acid may be formed using an organic base such as a primary, secondary, tertiary, or quaternary amine.

The polycarboxylic acid-comprising chelator compound is present in the solid composition at relatively high concentrations (on a weight basis) while the composition remains surprisingly nonfrangible. The minimum effective amount of chelator compound in a composition is related to the number of carboxyl groups in the chelator compound Succinic acid with two carboxylic acid is more efficacious than glutamic acid having same carboxylic acid groups since in glutamic acid COOH is zwitterion with —NH2.

Mucic acid is another example where 2 carboxylic acid groups are present but not as efficacious as succinic acid since COOH groups are further apart, sterically hindered. In certain embodiments, efficacy of the composition can be improved by depositing higher amount of dried composition. Efficacy is dependent on amount of acid in composition as well as amount of composition deposited.

Thus, in some embodiments, the chelator compound comprises at least about 5% of the dry essentially solvent free composition on a weight basis. In some embodiments, the chelator compound comprises at least about 10% of the essentially dry composition on a weight basis. In some embodiments, the chelator compound comprises at least about 15% of the essentially dry composition on a weight basis. In some embodiments, the chelator compound comprises at least about 20% of the essentially dry composition on a weight basis. In some embodiments, the chelator compound comprises at least about 25% of the essentially dry composition on a weight basis. In some embodiments, the chelator compound comprises at least about 30% of the essentially dry composition on a weight basis. In some embodiments, the chelator compound comprises at least about 35% of the composition on a weight basis. In some embodiments, the chelator compound comprises at least about 40% of the essentially dry composition on a weight basis. In some embodiments, the chelator compound comprises at least about 45% of the essentially dry composition on a weight basis. In some embodiments, the chelator compound comprises at least about 50% of the composition on a weight basis. In some embodiments, the chelator compound comprises at least about 55% of the essentially dry composition on a weight basis.

The term “essentially dry” or “essentially solvent free” is understood to mean a composition that has been processed to remove most of the solvent or has been processed in such a way that no solvent was required. This is generally the article for sale, e.g. before it has been applied to a patient. Generally, solvents are relatively volatile compounds having a boiling point at 760 mmHg ambient pressure of less than 150° C. and are used to process the composition but are removed to produce the final article for sale. For example, certain precursor compositions are first combined with water as a vehicle to form a solution, emulsion, or dispersion. This precursor composition is coated and dried on a substrate such that the water content of the coating is less than 10% wt/wt, preferably less than 5% wt/wt, and most preferably less than 2% wt/wt.

In some embodiments, the chelator compound comprises up to about 15% of the essentially dry composition on a weight basis. In some embodiments, the chelator compound comprises up to about 20% of the essentially dry composition on a weight basis. In some embodiments, the chelator compound comprises up to about 25% of the essentially dry composition on a weight basis. In some embodiments, the chelator compound comprises up to about 30% of the essentially dry composition on a weight basis. In some embodiments, the chelator compound comprises up to about 35% of the essentially dry composition on a weight basis. In some embodiments, the chelator compound comprises up to about 40% of the essentially dry composition on a weight basis. In some embodiments, the chelator compound comprises up to about 45% of the essentially dry composition on a weight basis. In some embodiments, the chelator compound comprises up to about 50% of the essentially dry composition on a weight basis. In some embodiments, the chelator compound comprises up to about 55% of the essentially dry composition on a weight basis. In some embodiments, the chelator compound comprises up to about 60% of the essentially dry composition on a weight basis.

In certain embodiments, wherein the polycarboxylic acid-comprising chelator compound comprises two aliphatic carboxylic acid groups (e.g., succinic acid), the chelator compound comprises at least about 10% of the essentially dry composition on a weight basis. In certain embodiments, wherein the polycarboxylic acid-comprising chelator compound comprises three aliphatic carboxylic acid groups (e.g., citric acid), the chelator compound comprises at least about 10% of the essentially dry composition on a weight basis. In certain embodiments, wherein the polycarboxylic acid-comprising chelator compound comprises four aliphatic carboxylic acid groups (e.g., ethylenediamine tetraacetic acid), the chelator compound comprises at least about 5% of the essentially dry composition on a weight basis.

When preparing compositions of the present disclosure, the polycarboxylic acid-containing chelator compound is dissolved and/or dispersed in a water-soluble plasticizer component and optionally a solvent such as water. The plasticizer component has a boiling point greater than 105 degrees C. and has a formula weight of less than 5000 atomic mass units. Preferably, the plasticizer component is a liquid at ambient temperature (23 degrees C.). Typically but not necessarily the plasticizer component is the most abundant solvent in the composition in which the polycarboxylic acid-containing chelator compound is dissolved and/or dispersed. In certain embodiments wherein water is used to prepare the composition, substantially all of the water is subsequently removed (e.g., after the composition has been coated onto a substrate.

In certain embodiments, the chelator compound comprises an aliphatic and/or aromatic polycarboxylic acid, in which two or more of the carboxylic groups are available for chelation without any zwitterionic interaction. Although potential zwitterionic interactions (e.g., such as in L-glutamic acid) may decrease antimicrobial efficacy relative to similar compounds (e.g., glutaric acid, succinic acid) that do not comprise a-amino groups, such zwitterionic compounds have been shown to exhibit antimicrobial activity in compositions according to the present disclosure. In addition, two or more carboxylic acid groups in the polycarboxylic acid-containing chelator compounds should be disposed in the chelator compound in sufficient proximity to each other or the compound should be capable of folding/conforming to bring the carboxylic acids sufficiently close to facilitate chelation of metal ions.

In certain embodiments, the chelator compound comprises an aliphatic polycarboxylic acid or a salt thereof, an aromatic polycarboxylic acid or a salt thereof, or a combination thereof In certain embodiments, the chelator compound comprises an aliphatic portion. In certain embodiments, the chelator compound comprises an aliphatic portion. The carboxylic acids may be disposed on the aliphatic portion and/or on the aromatic portion. Nonlimiting examples of chelator compounds that comprise an aliphatic portion with a carboxylic acid group disposed thereon and an aromatic portion with a carboxylic acid group disposed therein include 3-(2-Carboxyphenyl)propionic acid, 3-(4-Carboxyphenyl) propionic acid, and 4-[(2-Carboxyphenyl) amino]benzoic acid.

In certain embodiments, efficacy of the composition can be improved by depositing higher amount of dried composition. Efficacy is dependent on amount of acid in composition as well as amount of composition.

Suitable plasticizer components of the composition of the present disclosure include, but are not limited to, glycerol, a polyglycerol having 2-20 glycerin units, polyglycerols partially esterified with C1-C18 alkyl carboxylic acids having at least two free hydroxyl groups (e.g., hexaglycerol monolaurate, decaglycerol monolaurate, polyglyceryl-6 caprate, polyglyceryl-4 oleate, polyglyceryl-trilaurate and the like), polyethylene oxide, polyethylene glycol, polyethylene glycols initiated by any of the glycols discussed herein such as polyethylene glycol glyceryl ether, propylene glycol, dipropylene glycol, tripropylene glycol, 2-methyl 1,3 propane diol, sorbitol, dimethylisosorbide, pentaerythritol, trimethylol propane, ditrimethylolpropane, a random EO/PO copolymer or oligomer, a block EO/PO copolymer or oligomer, and a combination of any two or more of the foregoing plasticizer components.

The plasticizer component is present in the solid composition at relatively high concentrations (on a weight basis). In some embodiments, the plasticizer component comprises at least about 10% of the composition on a weight basis. In some embodiments, the plasticizer component comprises at least about 15% of the composition on a weight basis. In some embodiments, the plasticizer component comprises at least about 20% of the composition on a weight basis. In some embodiments, the plasticizer component comprises at least about 25% of the composition on a weight basis. In some embodiments, the plasticizer component comprises at least about 30% of the composition on a weight basis. In some embodiments, the plasticizer component comprises at least about 35% of the composition on a weight basis. In some embodiments, the plasticizer component comprises at least about 40% of the composition on a weight basis. In some embodiments, the plasticizer component comprises at least about 45% of the composition on a weight basis. In some embodiments, the plasticizer component comprises at least about 50% of the composition on a weight basis. In some embodiments, the plasticizer component comprises at least about 55% of the composition on a weight basis. In some embodiments, the plasticizer component comprises at least about 60% of the composition on a weight basis. In some embodiments, the plasticizer component comprises at least about 65% of the composition on a weight basis. In some embodiments, the plasticizer component comprises at least about 70% of the composition on a weight basis.

In some embodiments, the plasticizer component comprises up to about 20% of the composition on a weight basis. In some embodiments, the plasticizer component comprises up to about 25% of the composition on a weight basis. In some embodiments, the plasticizer component comprises up to about 30% of the composition on a weight basis. In some embodiments, the plasticizer component comprises up to about 35% of the composition on a weight basis. In some embodiments, the plasticizer component comprises up to about 40% of the composition on a weight basis. In some embodiments, the plasticizer component comprises up to about 45% of the composition on a weight basis. In some embodiments, the plasticizer component comprises up to about 50% of the composition on a weight basis. In some embodiments, the plasticizer component comprises up to about 55% of the composition on a weight basis. In some embodiments, the plasticizer component comprises up to about 60% of the composition on a weight basis. In some embodiments, the plasticizer component comprises up to about 65% of the composition on a weight basis. In some embodiments, the plasticizer component comprises up to about 70% of the composition on a weight basis. In some embodiments, the plasticizer component comprises up to about 75% of the composition on a weight basis.

In certain embodiments, the plasticizer component can act as a humectant. Advantageously, this can maintain a moist environment in a wound to help promote healing of the wound tissue. In these and other embodiments, articles comprising the composition can optionally be packaged in a moisture barrier package such as a foil package or any of the non-foil moisture-barrier packaging options disclosed in U.S. Pat. No. 8,105,306; which is incorporated herein by reference in its entirety.

Advantageously, the relatively high concentration of plasticizer and/or water-soluble or water-dispersible polymer in the composition can function as a controlled-release modulator that facilitates delivery of the antimicrobial(s) over an extended period of time. In some embodiments, the plasticizer component can function as an antimicrobial component.

Compositions according to the present disclosure are solid at 25 degrees C. In certain embodiments, the composition may comprise a solvent having a boiling point of less than or equal to 100 degrees C. Nonlimiting examples of such solvents include water and lower (C2-05) alcohols. Preferably, before use, the composition comprises very little solvent (e.g., less than or equal to about 10% by weight) having a boiling point of less than or equal to 100 degrees C. In some embodiments, the composition comprises less than 5%, less than 4%, less than 3%, less than 2%, or even less than 1% (by weight) of a solvent having a boiling point of less than or equal to 100 degrees C. In certain embodiments, the composition may be substantially free (before use) of such solvents or any compounds having a boiling point less than 100 C.

Compositions of the present disclosure comprise a water-soluble or water-dispersible polymer. The water-soluble or water-dispersible polymer has a T_{G} greater than or equal to 20 degrees C. In use, the polymer can function to form the composition into a cohesive shape such as a film while also absorbing wound exudate and to maintain a moist environment that can facilitate healing of the tissue at a wound site.

Nonlimiting examples of water-soluble or water-dispersible polymers that are suitable for use in a composition according to the present disclosure include a polyvinylpyrrolidone, a polyvinyl alcohol, butyene diol vinyl alcohol and its copolymers, polysaccharides such as starch, guar gum, locust bean gum, carrageenan, hyaluronic acid, agar, alginate, tragacanth, gum arabic, gum karraya, gellan, and xanthan gums as well as modifications of these such as hydroxyethyl-, hydroxypropyl-, or cationic derivatives; a modified cellulose polymer (e.g., hydroxyethylcellulose, hydroxypropyl methylcellulose, carboxymethylcellulose, cationic cellulose such as polyquaterium 4, and the like), a copolymer of polyvinylpyrrolidone and vinyl acetate, water soluble and water swellable polyacrylates (e.g. based on hydroxyethylacrylate, hydroxypropyl acrylate, acrylic acid, acrylamide, PEG acrylates, methyl acrlayte, methacrylates, and the like) and a combination of any two or more of the foregoing water-soluble or water-dispersible polymers. In certain embodiments, the water-soluble or water-dispersible polymers can comprise a polyquaternium polymer

In some embodiments, the water-soluble or water-dispersible polymer comprises at least about 5% of the composition on a weight basis. In some embodiments, the water-soluble or water-dispersible polymer comprises up to about 65% of the composition on a weight basis.

In certain embodiments, the antimicrobial activity of the chelator compound can be supplemented by adding to the composition an optional antimicrobial component. Optional antimicrobial components that are suitable for use in a composition according to the present disclosure include, but are not limited to, an antibiotic, the antiseptics disclosed in US20180207122 incorporated herein by reference, as well as other suitable antimicrobials. Preferred additional antimicrobials include an antimicrobial lipid, a phenolic antiseptic, a cationic antiseptic, iodine and/or an iodophor, a peroxide antiseptic, an antimicrobial natural oil, a C6-C12 alkane diol, silver, silver salts and complexes, silver oxide, copper, copper salts, or combinations thereof. Preferred additional antimicrobial compounds include antimicrobial quaternary amine compound (e.g., benzalkonium chloride) or a salt thereof, a cationic surfactant (e.g. cetylpyridinium chloride, cetyltrimethylammonium bromide, etc.), polycationic compounds such as octenidine or a salt thereof, a biguanide compound (e.g., Chlorhexidine, polyhexamethylenebiguanide (PHMB) or a salt thereof, a (C6-C12) 1,2-organic diol (e.g., 1,2-octanediol), an antimicrobial fatty acid monoester compound, and a combination of any two or more of the foregoing antimicrobial components.

In certain embodiments, the use of antimicrobial quaternary amine compound (e.g., benzalkonium chloride) or a salt thereof, when added to the composition may show better quality film than without them.

Many of the compositions have exceptional broad-spectrum antimicrobial activity and thus are generally not terminally sterilized but if necessary, may be sterilized by a variety of industry standard techniques. For example, it may be preferred to sterilize the compositions in their final packaged form using electron beam. It may also be possible to sterilize the sample by gamma radiation, nitrogen dioxide sterilization or heat. Other forms of sterilization may be acceptable. It may also be suitable to include preservatives in the formulation to prevent growth of certain organisms. Suitable preservatives include industry standard compounds such as Parabens (methyl, ethyl, propyl, isopropyl, isobutyl, etc.), 2 bromo-2 nitro-1,3 diol; 5 bromo-5-nitro-1,3 dioxane, chlorbutanol, diazolidinyl urea; iodopropylnyl butylcarbamate, phenoxyethanol, halogenated cresols, methylchloroisothiazolinone and the like, as well as combinations of these compounds. An antimicrobial quaternary amine compound (e.g., benzalkonium chloride) or a salt thereof, a cationic surfactant (e.g., octenidine) or a salt thereof, a biguanide compound (e.g., PHMB) or a salt thereof, a (C6-C12) 1,2-organic diol (e.g., 1,2-octanediol), an antimicrobial fatty acid monoester compound, and a combination of any two or more of the foregoing antimicrobial components may act as preservative as well.

The compositions adhere well to mammalian tissues (particularly, skin, mucosal tissue, and wounds), in order to deliver the antimicrobial to the intended site over a prolonged period even in the presence of perspiration, drainage (e.g., mucosal secretions), or mild lavage. In use, the compositions are typically non-aqueous.

Compositions can be delivered using a variety of techniques. Typically, the compositions are delivered to the skin and/or mucosal tissue in a manner that allows them to penetrate into the skin and/or mucosal tissue, as opposed to through the tissue into the blood stream. This concentrates the compositions locally at the site in need of treatment.

When contacting a wound site, the composition and/or articles of the present disclosure are hydrated by the tissue fluids and wound exudate. Compositions according to the present disclosure comprise polycarboxylic acid chelator compounds that, in an aqueous environment, have antimicrobial properties at an acidic pH. Thus, compositions of the present disclosure comprise appropriate quantities of acidic components (e.g., the free acid of the polycarboxylic acid chelator compound) and basic components (e.g., NaOH or a salt of the polycarboxylic acid chelator compound) such that the composition, when mixed well with deionized water at a 1:9 mass ratio, forms an aqueous mixture having a pH of about 2.5 to 5.5. In certain embodiments, the pH of the resulting aqueous mixture is at least 2.5. In certain embodiments, the pH of the resulting aqueous mixture is at least 3.0. In certain embodiments, the pH of the resulting aqueous mixture is at least 3.5. In certain embodiments, the pH of the resulting aqueous mixture is at least 4.0. In certain embodiments, the pH of the resulting aqueous mixture is at least 4.5. In certain embodiments, the pH of the resulting aqueous mixture is up to about 3.0. In certain embodiments, the pH of the resulting aqueous mixture is up to about 3.5. In certain embodiments, the pH of the resulting aqueous mixture is up to about 4.0. In certain embodiments, the pH of the resulting aqueous mixture is up to about 4.5. In certain embodiments, the pH of the resulting aqueous mixture is up to about 5.0. In certain embodiments, the pH of the resulting aqueous mixture is up to about 5.5.

A variety of other ingredients may be added to the antiseptic compositions for desired effect. These include, but are not limited to, surfactants, skin emollients and humectants such as those described in U.S. Pat. No. 5,951,993 (Scholz et al.), fragrances, colorants, tackifiers, plasticizers, etc. Other active agents that may be delivered to the skin using a composition include components of cosmetic compositions. These include, but are not limited to, vitamins, herbal extracts, antioxidants, steroids or other anti-inflammatory agents, vasodilators, chemitactic compounds, exfoliants such as alpha-hydroxy acids or beta-hydroxy acids, growth factors, enzymes, bleaching or coloring agents, emulsifiers, skin soothing agents, skin tightening agents, anti-wrinkle agents, skin repair agents, sebum inhibiting agents, sebum stimulators, protease inhibitors, anti-itch ingredients, agents for inhibiting hair growth, agents for accelerating hair growth, skin sensates, antiacne treatments, depilating agents, astringents, hair removers, or corn, callus or wart removers, decorative agents such as glitters, fragrances including aromatherapy agents, perfumes, sunscreen agents, insect repellants, deodorants and antiperspirants, hair colorants, bleaching agents, antidandruff agents. Various combinations of active agents can be used in the compositions.

In another aspect, the present disclosure provides an article. The article comprises a solid composition made from any embodiment of an antimicrobial composition according to the present disclosure. In some embodiments, the article comprises a thin film of the composition. The film may be sheet-like and, optionally, may be disposed on a surface (e.g., onto a substrate, medical dressing, or medical device as discussed herein). The article can be made, for example, by blending (e.g., in a homogenizer) the components and coating (e.g., knife-coating, spraying, die slot coating, dip coating, curtain coating, extruding the resulting formulation onto a surface. In some embodiments, a solvent such as water may be added to the ingredients to facilitate a coating process. Optionally, the coating formulation can be dried to remove excess water. In some embodiments wherein the plasticized polymer behaves as a thermoplastic, the composition may be processed without solvent using an extruder such as a twin screw extruder.

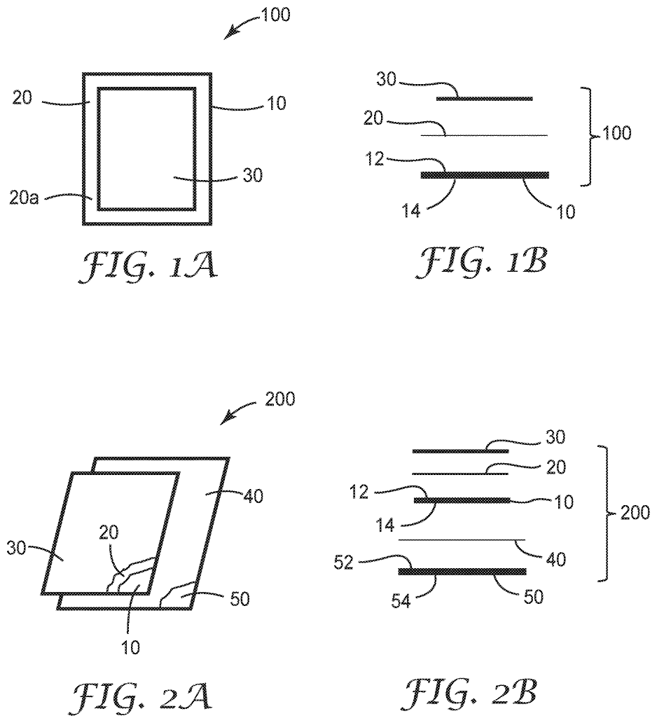

Turning to the drawings, FIGS. 1A and 1B show various views of one embodiment of an article 100 according to the present disclosure. The article 100 comprises a substrate 10 having a first major surface 12 and a second major surface 14 opposite the first major surface. In addition, the article 100 comprises at least one layer (e.g., layer 30) adhered to the first major surface 12. In some embodiments, the at least one layer 30 comprises any embodiment of the antimicrobial composition according to the present disclosure. In some embodiments, the at least one layer 30 consists essentially of any embodiment of the antimicrobial composition according to the present disclosure. In some embodiments, the at least one layer 30 consists any embodiment of the antimicrobial composition according to the present disclosure.

In some embodiments, the at least one layer 30 adheres directly to the substrate 10. Optionally, the article 100 can comprise a first adhesive 20 adhered to at least a portion (e.g., at least 5%, at least 10%, at least 20%, at least 30%, at least 40%, at least 50%, at least 60%, at least 70%, at least 80%, at least 90% or 100%) of the surface area of first major surface 12. Thus, in some embodiments, the at least one layer 30 can be adhered to the first adhesive 20. In some embodiments, at least a portion of the first adhesive 20 (e.g., portion 20a, as shown in the illustrated embodiment of FIG. 1A) is not overlapped by the at least one layer 30. Thus, the exposed portion 20a of the first adhesive can be used to secure the article 100 to a surface (e.g., the skin adjacent or surrounding a wound site, not shown).

The substrate 10 can comprise any material suitable for use in a medical article. Suitable substrates include, but are not limited to, a fibrous material, a foam, a sheet material, a nonwoven material, a woven material, a solid polymeric material, a polymeric film, plastic, paper, molded fiber, rubber, glass, ceramic, metal, a metal foil, a surface of a medical device, and a combination of any two or more of the foregoing substrates. The dried composition can be placed in any arrangement with the substrate. There may be layers of dried combinations and fluid absorbing materials made from same material or different materials. Preferably, the substrate is nonlinting. The fluid absorbing material include cotton, rayon, carboxymethyl cellulose, acrylics, acetate fibers, alginates and other synthetic and natural polymers or blends. Silver coated fibers. Nonwoven fibrous wound dressings are being used more commonly nowadays in the management of highly exuding wounds. Carboxymethylcellulose (CMC) and alginate fibers are commonly used fibers. Foam material includes polyethylene foams, cross-linked polyethylene foams, polyurethane foams, reticulated polyurethane foams, melamine foams, etc.

In some embodiments, dried composition in form of film may be separately placed inside the wound. In some embodiments, dried composition in form of film and fluid absorbing material may be separately placed inside the wound.

It is contemplated that, when the substrate on which the composition is disposed is a portion of a medical device (e.g., a catheter), the first major surface on which the composition is disposed (e.g., as a coating) may be a contoured surface (i.e., not necessarily a generally flat, planar surface) and that the substrate may not comprise a second major surface opposite the first major surface.

In certain embodiments, the substrate is porous prior to coating. It is contemplated that the composition of the present disclosure can be disposed on the surface of a porous substrate as well as inside (e.g., at least partially inside) the pores and may extend completely through the pores.

In some embodiments, articles comprising a porous substrate can be secured to a patient (e.g., at a wound site) using a medical tape or a transparent adhesive dressing, for example.

FIGS. 2A and 2B show an alternative embodiment of an article 200 comprising a porous substrate. The article 200 comprises a substrate 10, optional first adhesive 20 and the at least one layer 30 as described hereinabove for the article 100 of FIGS. 1A-B. In addition, the article 200 comprises a backing layer 50 having a first side 52 and a second side 54 opposite the first side. In addition, the second major surface 14 of the substrate 10 is adhered (e.g., via optional second adhesive 40) to the first side 52 of the backing layer 50. The optional second adhesive 20 is adhered to at least a portion (e.g., at least 5%, at least 10%, at least 20%, at least 30%, at least 40%, at least 50%, at least 60%, at least 70%, at least 80%, at least 90% or 100% of the surface area) of the first side 52 of the backing layer 50. In some embodiments, at least a portion (not shown) of the second adhesive 40 is not overlapped by the substrate 10. Thus, the exposed portion of the second adhesive can be used to secure the article 200 to a surface (e.g., the skin adjacent or surrounding a wound site, not shown).

The backing layer is preferably a barrier to liquid and bacteria but is a high moisture vapor permeable film such as described in U.S. Pat. Nos. 3,645,835 and 4,595,001, the disclosures of which are herein incorporated by reference. In one embodiment, the backing layer is comprised of an elastomeric polyurethane, polyester, or polyether block amide films. These films combine the desirable properties of resiliency, elasticity, high moisture vapor permeability, and transparency. A description of this characteristic of materials for constructing the backing layer can be found in issued U.S. Pat. Nos. 5,088,483 and 5,160,315, the disclosures of which are hereby incorporated by reference. Commercially available examples of potentially suitable sheet materials for the backing layer may include the thin polymeric film backings sold under the trade names TEGADERM (3M Company), OPSITE (Smith & Nephew), etc. Many other backing layer materials may also be used, including those commonly used in the manufacture of surgical incise drapes (e.g., incise drapes manufactured by 3M Company under the trade names STERIDRAPE and IOBAN), etc. as described in U.S. Pat. No. 5,985,395, which is incorporated herein by reference in its entirety.

In certain embodiments, the substrate (e.g., a polymeric film, a woven material, a nonwoven material) on which the composition is disposed as a layer is flexible. Thus, an article comprising a substrate with a flexible layer of composition of the present disclosure disposed thereon advantageously can be applied to and conform to the shape of an irregular (e.g., curved, angular, cratered, bumpy) surface that might be found on a patient's body or on a medical device. In certain embodiments, the layer of composition disposed on the substrate of the article is at least as flexible as the substrate of the article.

In some embodiments, the at least one layer is coated relatively thin (e.g., less than about 5 mm, less than about 4 mm, less than about 3 mm, less than about 2 mm, or less than about 1 mm) of the essentially dry antimicrobial composition is substantially optically transparent and advantageously permits observation and inspection of objects (e.g., wound tissue, medical devices) disposed beneath the layer. Transparency is an indication of the compatibility of the components of the composition and can be assessed by coating the composition onto a suitable flat release liner, drying the composition, and assessing the percent transmission. This is described in the examples. In order to make a meaningful comparison the composition should be coated on an optically flat release liner at a specified thickness, dried appropriately, and maintained dry until testing. Compatibility and transparency may be affected by various factors including pH which determines the extent of ionization of the chelator, addition of a surfactant such as those disclosed in U.S. Pat. No. 8,512,723 incorporated herein by reference as well as antimicrobial surfactants such as benzalkonium chloride, cetylpyridinium chloride, and the like, and the type and amount of plasticizer. Finally, film thickness and film roughness can affect % transmission so the film should be kept relatively thin, e.g. <5mm and an optically flat liner is preferred.

In certain embodiments, the substrate (e.g., a polymeric film) on which the composition is disposed as a layer is substantially optically transparent. Thus, an article comprising an optically transparent substrate with an optically transparent layer of the composition of the present disclosure disposed (e.g., coated) thereon advantageously provides visual inspection of objects (e.g., wound tissue, medical devices) disposed beneath the article. Preferred compositions allow a caregiver to observe a wound over which a dressing comprising the composition is applied without removing the dressing. Preferred dressings are transparent both initially and after absorbing clear wound fluid.

In some embodiments, articles of the composition that form a sheet or a layer (e.g., less than about 3 mm, less than about 2 mm, less than about 1 mm, less than about 0.5mm, or less than about 0. mm is flexible (i.e., is able to conformed to irregular surfaces without cracking and/or flaking to an extent that causes disintegration of the sheet or layer). Advantageously, this permits application of the articles to irregular (e.g., curved, angular, cratered, bumpy) surfaces that might be found on a patient's body or on a medical device.

In certain embodiments, a composition according to the present disclosure can be disposed (e.g., as a layer or coating) on a medical device. The medical devices include medical devices on which microorganisms may form colonies or biofilms while the medical device is resident on or in a patient. Nonlimiting examples of such medical devices include a venous or urinary catheter, a cannula, a tracheotomy tube, an ostomy flange, an ostomy gasket, and an ostomy bag.

Compositions when coated and dried are flexible, meaning a thin film of one of these compositions, when bent over itself does not break or shatter when this testing is done immediately after following the drying and thin film is still in a film form.

In yet another aspect, the present disclosure provides a method of treating or preventing formation of a biofilm (e.g., in a wound site or on a medical device).

From a microbiological perspective, the primary function of normal, intact human and animal skin is to control microbial populations that live on the skin surface and to prevent underlying tissue from becoming colonized and invaded by potential pathogens. Exposure of subcutaneous tissue (i.e. a wound) provides a moist, warm and nutritious environment that is conducive to microbial colonization and proliferation. Since wound colonization is mostly polymicrobial, involving numerous microorganisms that are potentially pathogenic, any wound is at some risk of becoming infected. If an infection in a wound fails to heal, the patient suffers increased trauma as well as increased treatment costs.

Most wound infections are caused by Staphylococcus aureus (20%), Staphylococcus epidermidis (14%), Enterococci spp. (12%), Escherichia coli (8%), Pseudomonas aeurginosca (8%), Enterobacter spp. (7%), Proteus spp. (3%), Klebsielia pneumoniae (3%), Streptococci (3%) and Candida albicans (3%). Wound healing and infection is influenced by the relationship between the ability of bacteria to create a stable community within a wound environment and the ability of the host to control the bacterial community. Since bacteria are rapidly able to form their own protective microenvironment (biofilm) following their attachment to a surface, the ability of the host to control these organisms is likely to decrease as the biofilm community matures. Within a stable biofilm commnunity, interactions between aerobic and anaerobic bacteria are likely to increase their net pathogenic effect, enhancing their potential to cause infection and delay healing.

A method according to the present disclosure comprises contacting a tissue with any embodiment of a composition according to the present disclosure. Contacting the tissue with the composition further can comprise covering the composition and the tissue with a protective layer (e.g., a tape, a dressing).

Alternatively, a method according to the present disclosure comprises contacting a tissue with an article comprising any embodiment of the composition according to the present disclosure. The article comprising the composition can further comprise a substrate wherein the composition is disposed as a layer on and/or in the substrate as disclosed herein.

In certain embodiments, contacting a tissue with the composition comprises contacting a wound site (e.g., an acute wound, a chronic wound, a surgical wound, a site at which a medical device such as a needle or wire is inserted percutaneously) with the composition.

In certain embodiments, contacting a tissue with an article comprising the composition (e.g., a medical device coated with the composition) comprises contacting the tissue with a medical device comprising the composition. Alternatively, the composition may be coated and dried directly on a medical device. Nonlimiting examples of such medical devices include a venous or urinary catheter, a cannula, a tracheotomy tube, a nasogastric tube, surgical tools including but not limited to colonoscopes, cystoscopes, laproscopes, bronchoscopes and the like, an ostomy flange, an ostomy gasket, an ostomy bag, and oral implants.

Contacting a tissue with the composition or an article comprising the composition (e.g., as a coating) comprises contacting a tissue with the composition for a period of time. The period of time is preferably about 0.5 hours to about 72 hours.

In certain preferred embodiments, an article (e.g., a thin film or a coated substrate) is attached to wound fluid-absorbing material (e.g., a medical dressing), which is then placed over the wound site.

Objects and advantages are further illustrated by the following examples, but the particular materials and amounts thereof recited in these examples, as well as other conditions and details, should not be construed to unduly limit this invention.

### EXAMPLES

### Materials

TABLE 1
Materials used in the Examples

| Ingredient | Name | Supplier |
|---|---|---|
| Glycerol | Glycerol | Cargill Corporation, |
| | | Wayzata, MN |
| Water Sterile | Water | Rocky Mountain Biologicals, |
| | | Inc. |
| | | Missoula, Montana |
| Sodium Citrate | Sodium Citrate | Sigma Aldrich |
| (trisodium salt dihydrate) | | |
| Citric Acid Monohydrate | Citric Acid | Sigma Aldrich |
| Linear | L-PVPK60 | Ashland Inc. |
| Polyvinylpyrrolidone K 60 | | |
| (47% in water) | | |
| Ethylenediaminetetraacetic | EDTA | Alfa Aesar; Haverhill, MA |
| acid dipotassium salt | | |
| dihydrate | | |
| Sodium Hydroxide | NaOH | VWR, Radnor, PA |
| (50% in water) | | |
| L-Tartaric Acid | Tartaric Acid | Sigma Aldrich |
| Succinic Acid | Succinic Acid | Sigma Aldrich |
| Hydrochloric acid 1N | HCl | VWR |
| DL-Malic Acid | Malic Acid | Alfa Aesar |
| Maleic acid | Maleic acid | Sigma-Aldrich |
| Gluconic acid (50% in water) | Gluconic acid | Sigma Aldrich |
| MPDiol ® glycol | 2-methyl-1,3-propanediol | |
| Dipropylene glycol | Dipropylene glycol | LyondellBasell, Houston, |
| | | Texas |
| Diethylene glycol | Diethylene glycol | Sigma Aldrich |
| Liponic EG-7 | Polyethylene glycol ether of | Lipo Chemical Inc, Paterson, |
| Glycereth-7 | glycerin | NJ |
| Arcol LG-650 | Arcol LG-650 polyether | Covestro LLC, Pittsburgh, |
| | polyol is a 260-molecular- | Pennsylvania |
| | weight polypropylene oxide- | |
| | based triol | |
| Glucam P-10 | propoxylated methyl glucose | Lubrizol Advanced Materials, |
| | ether | Inc. Cleveland, OH |
| Arlasolve DMI | Dimethyl Isosorbide | Croda Inc. |
| | | Edison, NJ |

Test Method for Anti-Biofilm Antimicrobial Activity: Ex Vivo Porcine Mucosal Tissue Biofilm Assay

Tissue Prep: Ex vivo porcine vaginal mucosal tissue was trimmed transferred into RPMI 1640 medium+5% penicillin/streptomycin solution (part #P4458 obtained from Sigma-Aldrich, St. Louis, Mo.). Biopsy punches (5 mm diameter) were prepared to produce the explants for this assay. Most of the remaining muscle tissue was removed with a fresh scalpel blade. The explants were rinsed three times with 10±2 ml RPMI (no antibiotics, no Fetal Calf Serum). Explants were covered with fresh media placed in incubator for ˜30 min. at 37° C. A 6-well plate was prepared with 2.0±0.5 mL RPMI (no antibiotics, no Fetal Calf Serum) in the wells and a transwell insert was placed in each well. Tissue explants were transferred mucosal side-up to the transwell inserts (3 explants/well).

Bacteria Prep: A biofilm-producing strain of Pseudomonas aeruginosa was used for these experiments. A fresh agar culture of each bacterial strain from frozen stock was prepared within two weeks of the experiment. A culture tube containing Todd Hewitt broth was inoculated with several colonies and placed into a shaking incubator, (37±2° C., 200±50 rpm) overnight. A 1±0.1 mL portion of the overnight culture was removed from the overnight culture and placed into a sterile microcentrifuge tube. The microcentrifuge tube was centrifuged (1±0.5 min at max speed) to pellet the bacteria. The pellet was washed with 1±0.1 mL RPMI, no ABX, no FCS. After washing, the pellet was resuspended in 1±0.1 mL of fresh RPMI. A 300±20 μl portion of the resuspended cells was diluted into 5±0.5 mL of fresh RPMI. The resulting diluted bacterial suspensions (2±1 μl per explant) were added to each transwell insert and the 6-well plates were returned to the 37° C. incubator for 2±0.5 hr. to infect the explants.

Treatment: Antimicrobial compositions (a 10 mm by 10 mm piece of the dried antimicrobial films described below) were applied directly to the microorganism-seeded mucosal tissue (explant). After applying the dried films, the microwell plates were incubated at 37±2° C. for 24±4 h.

Sampling: The explants were transferred into 250±20 μL Standard Sampling Solution and then vortex mixed for 30±10 seconds, sonicated briefly to disrupt cell aggregates, and then vortex mixed for another 30±10 seconds. A portion of the resulting sonicates were stored at 4 degrees C. Another portion of the sonicates were plated at appropriate dilutions on cetrimide selective agar, incubated overnight and colonies were counted. If the initial colony counts were too numerous to count, the refrigerated portions of the sonicates were diluted further, plated on cetrimide agar, incubated, and colonies were counted after the incubation.

### Examples 1-12. Antimicrobial Compositions Comprising Glycerol as the Water-Soluble Plasticizer Component

Preparation of Compositions:

All compositions were made in 100 g quantities according to the formulae listed in Table 2. A mixture of L-PVPK60 (47 wt % in water) was made. All compositions shown in Table 2 comprised 50 g of the aforementioned aqueous mixture of L-PVPK60 in addition to the components listed in the Table. For each composition, all of the ingredients except the L-PVPK60 were added to a MAX 100 cup (Flacktec Inc.; Landrum, S. C.) and mixed at 3500 rpm for 1 minute using a DAC 400 FVZ SpeedMixer™ instrument (Flacktec, Inc.). Subsequently, 50 g of the L-PVPK60 aqueous mixture was added to the cup and mixed for an additional minute at 3500 rpm.

Preparation of Articles Comprising the Compositions:

On the day each composition was produced as described above, the viscous composition was knife-coated onto a release liner using gaps of 127 microns, 254 microns, 381 microns, 508 microns, 635 microns, 762 microns, 1016 microns, and 1270 microns, respectively, in order to produce films having various thicknesses. The coatings were dried at 80 degrees C. for 10 minutes in a convection oven to remove substantially all of the water from the coating. The dried antimicrobial film was peeled off the liner and was tested for anti-biofilm antimicrobial activity as described above.

The results of the antimicrobial testing are shown in Table 3.

TABLE 2
Components of the antimicrobial compositions of Examples 1-13.

| | L-PVPK60 | | | | | | | | |
|---|---|---|---|---|---|---|---|---|---|
| | Mixture | | | | | | | Total | |
| Example | (47% in | Glycerol | Water | | Acid | | Base | Chelator | |
| No. | water) (g) | (g) | (g) | Acid | (Wt %) | Base | Wt % | (Wt %) | pH |
| 1 | 60.06 | 22.85 | 0 | Citric | 10.94 | Sodium | 14.58 | 21.79 | 4.45 |
| | | | | acid | | citrate | | | |
| 2 | 53.66 | 20.49 | 1.81 | Citric | 22.02 | NaOH¹ | 7.52 | 22.02 | 3.87 |
| | | | | acid | | | | | |
| 3 | 59.53 | 22.73 | 0.0 | Tartaric | 23.47 | NaOH | 1.4 | 23.47 | 3.6 |
| | | | | acid | | | | | |
| 4 | 60.28 | 22.91 | 0.0 | Tartaric | 16.43 | NaOH | 4.72 | 16.43 | 5.32 |
| | | | | acid | | | | | |
| 5 | 58.42 | 25.33 | 7.95 | Succinic | 12.51 | NaOH | 0.38 | 12.51 | 3.13 |
| | | (Polyglycerol-3) | | acid | | | | | |
| 6 | 57.71 | 23.1 | 4.00 | Malic | 15.37 | NaOH | 4.44 | 15.37 | 4.47 |
| | | | | acid | | | | | |
| 7 | 50.59 | 19.22 | 10.28 | Malic | 22.2 | NaOH | 5.61 | 22.2 | 4.04 |
| | | | | acid | | | | | |
| 8 | 57.8 | 21.97 | 0.0 | Succinic | 12.5 | Sodium | 16.67 | 24.90 | 4.55 |
| | | | | acid | | citrate | | | |
| 9 | 51.81 | 19.69 | 10.36 | Tartaric | 12.19 | Sodium | 16.25 | 24.28 | 3.79 |
| | | | | acid | | citrate | | | |
| 10 | 66.6 | 25.35 | 0.0 | EDTA | 12.44 | None | 0 | 12.44 | 4.76 |
| 11 | 65.07 | 24.76 | 0.0 | Citric | 11.73 | NaOH | 1.75 | 11.73 | 3.49 |
| | | | | acid | | | | | |
| 12 | 55 | 21.36 | 0.00 | Maleic | 14.64 | NaOH | 9.0 | 14.64 | 4.32 |
| | | | | acid | | | | | |

¹- 50% (w/w) NaOH in water

TABLE 3
Antimicrobial activity of the compositions of Examples 1-12.
The Log Reduction Value (LRV) was calculated by subtracting
the logic number of viable bacteria after exposure to the composition
from the log₁₀ number of viable bacteria before exposure to the
composition according to the method described herein.

| Example | LRV |
|---|---|
| 1 | 5.95 |
| 2 | 7.57 |
| 3 | 7.57 |
| 4 | 3.82 |
| 5 | 7.16 |
| 6 | 2.04 |
| 7 | 7.57 |
| 8 | 2.72 |
| 9 | 6.79 |
| 10 | 3.40 |
| 11 | 4.27 |
| 12 | 3.42 |

The data in Table 3 show that each of the compositions, with various polycarboxylic acid chelator compounds exhibited bactericidal activity against the microorganisms used in the biofilm test model.

### Comparative Examples 1-4. Effect of Concentration of Polycarboxylic Acid-Containing Chelator Compound

Preparation of Compositions:

All compositions were made in 100 g quantities according to the formulae listed in Table 4. A mixture of L-PVPK60 (47 wt % in water) was made. All compositions shown in Table 4 comprised 50 g of the aforementioned aqueous mixture of L-PVPK60 in addition to the components listed in the Table. For each composition, all of the ingredients except the L-PVPK60 were added to a MAX 100 cup (Flacktec Inc.; Landrum, S.C.) and mixed at 3500 rpm for 1 minute using a DAC 400 FVZ SpeedMixer™ instrument (Flacktec, Inc.). Subsequently, 50 g of the L-PVPK60 aqueous mixture was added to the cup and mixed for an additional minute at 3500 rpm.

Articles (i.e., thin films coated on a liner) of each composition were made as described for Examples 1-12 and were tested for antimicrobial activity as described hereinabove. WAS THE pH MEASURED? The results of the antimicrobial tests are shown in Table 5.

TABLE 4
Components of the compositions of Comparative Examples 1-4.

| Comparative | L-PVPK60 | | | | Sodium | Total |
|---|---|---|---|---|---|---|
| Example | Mixture | Glycerol | Water | Citric Acid | Citrate | Chelator |
| No. | (g) | (g) | (g) | (wt %) | (wt %) | (Wt %) |
| 1 | 69 | 26.16 | 2.65 | 1.55 | 2.06 | 3.07 |
| 2 | 71.6 | 27.2 | ?0.1 | 0.76 | 1.02 | 1.49 |
| 3 | 71.73 | 27.26 | 0.47 | 0.37 | 0.5 | 0.74 |
| 4 | 71.73 | 27.26 | 1.01 | 0.0 | 0.0 | 0.0 |

TABLE 5
Antimicrobial activity of the compositions of
Comparative Examples 1-4. The Log Reduction
Value (LRV) was calculated as described above.

| Comparative | |
|---|---|
| Example | LRV |
| 1 | <1 |
| 2 | <1 |
| 3 | <1 |
| 4 | <1 |

The data in Table 5 show that each of the compositions, with various, relatively low concentrations of polycarboxylic acid chelator compound, did not exhibit significant bactericidal activity against the microorganisms used in the biofilm test model.

### Examples 13-19. Antimicrobial Compositions Comprising Various Water-Soluble Plasticizer Components

Preparation of Compositions:

All compositions were made in 100 g quantities according to the formulae listed in Table 6. A mixture of L-PVPK60 (47 wt % in water) was made. All compositions shown in Table 6 comprised 50 g of the aforementioned aqueous mixture of L-PVPK60 in addition to the components listed in the

Table. For each composition, all of the ingredients except the L-PVPK60 were added to a MAX 100 cup (Flacktec Inc.; Landrum, SC) and mixed at 3500 rpm for 1 minute using a DAC 400 FVZ SpeedMixer™ instrument (Flacktec, Inc.). Subsequently, 50 g of the L-PVPK60 aqueous mixture was added to the cup and mixed for an additional minute at 3500 rpm.

Articles (i.e., thin films coated on a liner) of each composition were made as described for Examples 1-12 and were tested for antimicrobial activity as described hereinabove. The results of the antimicrobial testing are shown in Table 7.

TABLE 6
Components of the antimicrobial compositions of Examples 13-19.

| | | | | Citric | Sodium | Total | |
|---|---|---|---|---|---|---|---|
| Example | | Plasticizer | Water | Acid | Citrate | Chelator | |
| No. | Plasticizer | (g) | (g) | (g) | (g) | (Wt %) | pH |
| 13 | 2-Methyl-l,3- | 19.0 | 13.5 | 12.5 | 16.67 | 24.91 | 4.36 |
| | propanediol | | | | | | |
| 14 | Dipropylene | 19.0 | 13.5 | 12.5 | 16.67 | 24.91 | 4.34 |
| | glycol | | | | | | |
| 15 | Diethylene glycol | 19.0 | 13.5 | 12.5 | 16.67 | 24.91 | 4.40 |
| 16 | Glycereth-7 | 19.0 | 13.5 | 12.5 | 16.67 | 24.91 | 4.38 |
| 17 | Arcol LG-650 | 19.0 | 13.5 | 12.5 | 16.67 | 24.91 | 4.39 |
| 18 | Glucam P-10 | 19.0 | 13.5 | 12.5 | 16.67 | 24.91 | 4.40 |
| 19 | Arlamol ™ DMI | 19.0 | 13.5 | 12.5 | 16.67 | 24.91 | 4.33 |

TABLE 7
Antimicrobial activity of the compositions of
Examples 13-19. The Log Reduction Value
(LRV) was calculated as described above.

| Example | LRV |
|---|---|
| 13 | 8.01 |
| 14 | 7.22 |
| 15 | 4.06 |
| 16 | 5.11 |
| 17 | 7.34 |
| 18 | 8.01 |
| 19 | 2.66 |

The data in Table 7 show that each of the compositions, with various polycarboxylic acid chelator compounds exhibited bactericidal activity against the microorganisms used in the biofilm test model.

Flexibility and Optical Transparency Testing

Flexibility and % Transmittance testing was carried out on following example compositions:

| | | | | % | Standard |
|---|---|---|---|---|---|
| | Average | Standard | | Transmit- | Deviation |
| Example | Thickness | Deviation | Flex- | tance | % |
| No. | mm | mm | ibility | Average | Transmittance |
| 1 | 0.145 | 0.004 | Pass | 20.53 | 1.70 |
| 3 | 0.191 | 0.008 | Pass | 30.63 | 5.45 |
| 4 | 0.168 | 0.008 | Pass | 43.85 | 3.69 |
| 6 | 0.119 | 0.010 | Pass | 40.66 | 1.65 |
| 7 | 0.139 | 0.012 | Pass | 7.14 | 0.92 |
| 9 | 0.136 | 0.004 | Pass | 13.50 | 1.73 |
| 10 | 0.164 | 0.032 | Pass | 30.07 | 1.17 |
| 12 | 0.167 | 0.010 | Pass | 57.60 | 0.90 |
| 14 | 0.143 | 0.003 | Pass | 12.92 | 1.22 |
| 16 | 0.192 | 0.005 | Pass | 36.05 | 1.96 |
| 17 | 0.120 | 0.001 | Pass | 30.84 | 0.26 |
| 19 | 0.083 | 0.009 | Pass | 31.20 | 2.59 |

Flexibility Test: To access the flexibility of the films, films were coated on to the UV cured silicone coated liner having a thickness of 50 microns at a thickness of approximately 0.15 mm immediately after compounding the ingredients as described earlier. The films were dried at 80 degree centigrade for 10-20 minutes before being taken out of oven, allow to come to room temperature, and immediately tested for flexibility in a room having temperature 22-degree centigrade and relative humidity of 66%. The additional liner was placed on top of the thin film and 10.16 centimeter by 25.4-centimeter piece of thin film having liners on both sides was cut. The piece was then folded approximately in half at 180-degree angle and creased using middle finger of adult man moved once over crease. After this time, film along with liners on both sides was unfolded, top liner was taken off and film was visibly checked for breaking, shattering, or flaking. No visible breaking, shattering or flacking of film is termed as pass.

Transmittance Test: To access the % transmittance of the films, films were coated on to the liner immediately after compounding the ingredients as described earlier. The films were dried at 80 degree centigrade for 10-20 minutes before being taken out from oven, allowed to come to room temperature, and immediately tested for % transmittance in room having temperature 22-degree centigrade and relative humidity of 63%. The additional liner was placed on top of the thin film and 10.16 centimeter by 10.16-centimeter piece of thin film having liners on both sides was cut. Glass slide having dimensions 7.62 centimeter by 5.08 centimeter having thickness of 1.016 mm was placed on thin film after taking out the liner from one side of thin film and placing glass slide on to it. For measurement of % transmittance, liner from other side was peeled off and glass slide with thin film was placed perpendicular behind the cuvette in Hewlett Packard 8453 UV-VIS spectrophotometer. The reading was recorded at three different locations of the films and values are reported in % transmittance.

The complete disclosures of the patents, patent documents, and publications cited herein are incorporated by reference in their entirety as if each were individually incorporated. Various modifications and alterations will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. An article, comprising:
a substrate having a first major surface and, optionally, a second major surface opposite the first major surface; and
at least one layer comprising a composition, the at least one layer adhered to the first major surface;
the composition comprising:
a water-soluble plasticizer component characterized by a boiling point greater than 105° C. and has a formula weight of less than 5000 atomic mass units,
a dicarboxylic acid or tricarboxylic acid chelator component, or a salt thereof present in an amount of at least 10% (w/w) of the composition, or
a tetracarboxylic acid chelator component, or a salt thereof present in an amount of at least 5% (w/w) of the composition,
wherein the selected chelator component is mixed with the water-soluble plasticizer component, and
a water-soluble or water-dispersible polymer characterized by a T_{G} greater than or equal to 20° C. dissolved, wherein the water-soluble or water-dispersible polymer is optionally dispersed in the water-soluble plasticizer component,
wherein the composition comprises less than 10 wt % of a solvent that has a boiling point less than or equal to 100 degrees C.,
wherein the composition is a solid at 25° C. and
wherein the composition, when mixed with deionized water at a 1:9 mass ratio, forms an aqueous mixture having a pH of about 2.5-5.5.

2. The article of claim 1, wherein the chelator compound comprises an aliphatic polycarboxylic acid or a salt thereof, an aromatic polycarboxylic acid or a salt thereof, or a combination thereof.

3. (canceled)

4. The article of claim 2, wherein the chelator compound is selected from the group consisting of citric acid, succinic acid, tartaric acid, maleic acid, glutaric acid, malic acid, a salt thereof, a combination thereof, phthalic acid, trimesic acid, and a combination thereof.

5-7. (canceled)

8. The article of claim 1, wherein the chelator compound is ethylenediamine tetraacetic acid, pyromellitic acid, benzophenone tetracarboxylic acid, phthalic acid, or trimesic acid.

9-10. (canceled)

11. The article of claim 1, wherein the water-soluble or water-dispersible polymer is selected from the group consisting of a polyvinylpyrrolidone, a polyvinyl alcohol, butyene diol vinyl alcohol and its copolymers, a polysaccharide, a modified cellulose polymer, a copolymer of polyvinylpyrrolidone and vinyl acetate, a water soluble or water swellable polyacrylates, and a combination of any two or more of the foregoing water-soluble or water-dispersible polymers, and combinations thereof.

12. The article of claim 1, wherein the plasticizer component is selected from the group consisting of glycerol, a polyglycerol having 2-20 glycerin units, a polyglycerols partially esterified with a C1-C18 alkyl carboxylic acid having at least two free hydroxyl groups, polyethylene oxide, polyethylene glycol, propylene glycol, dipropylene glycol, tripropylene glycol, 2-methyl 1,3 propane diol, sorbitol, pentaerythritol, trimethylol propane, ditrimethylol propane, a random EO/PO copolymer or oligomer, and a block EO/PO copolymer or oligomer.

13. The article of claim 1, wherein the chelator compound is present in the composition up to about 60 wt %.

14. The article of claim 1, wherein the plasticizer component is present in the composition at about 10 wt % to about 75 wt %.

15. The article of claim 1, wherein the water-soluble or water-dispersible polymer is present in the composition at about 5 wt % to about 65 wt %.

16. The article of claim 1, wherein the composition further comprises an antimicrobial component.

17. The article of claim 16, wherein the antimicrobial component is selected from the group consisting of an antibiotic, an antimicrobial quaternary amine compound or a salt thereof-, a biguanide compound or a salt thereof, a (C6-C12) 1,-2-organic diol, an antimicrobial fatty acid monoester compound, a cationic surfactant, and a combination thereof.

18. The article of claim 1, wherein the composition is substantially water-free.

19. The article of claim 1, wherein the substrate is selected from the group consisting of a fibrous material, a foam, a sheet material, a nonwoven material, a woven material, a knitted material, a polymeric film, a surface of a medical device, and a combination of any two or more of the foregoing substrates.

20. The article of claim 1, wherein the layer has greater than 5% transmittance at 550 nm when tested according to the Transparency Test.

21-25. (canceled)

26. The article of claim 1, wherein the at least one layer is about 50 microns thick to about 5000 microns thick.

27. (canceled)

28. The article of claim 1, wherein the substrate is at least a part of a medical device selected from the group consisting of a catheter, a cannula, a tracheotomy tube, an ostomy flange, an ostomy gasket, an ostomy bag, and an oral implant.

29-32. (canceled)

33. A method of treating or preventing formation of a biofilm, the method comprising contacting a tissue with the at least one layer of the article of claim 1.

34. A method of treating a tissue to reduce a number of microorganisms residing therein or thereon, the method comprising contacting the tissue with the at least one layer of the article of claim 1.

35-36. (canceled)

37. A composition, comprising:
a chelator compound comprising:
a dicarboxylic acid or tricarboxylic acid chelator component, or a salt thereof,
wherein the dicarboxylic acid or the tricarboxylic acid
chelator component, or a salt thereof is present in an amount of at least about 10% (w/w) of the composition;
a water-soluble or water-dispersible polymer having a T_{G} greater than or equal to 20° C.;
a water-soluble plasticizer component having a boiling point greater than 105° C. and having a formula weight of less than 5000 atomic mass units; and
a solvent present in an amount of less than 10 wt % of the composition, wherein the solvent is characterized by a boiling point less than or equal to 100 degrees C.,
wherein the composition is a solid at 25° C., and
wherein the composition, when mixed with deionized water at a 1:9 mass ratio, forms an aqueous mixture having a pH of about 2.5-5.5.

38. A composition, comprising:
a chelator compound comprising:
a tetracarboxylic acid chelator component, or a salt thereof;
wherein the composition comprises at least about 5% (w/w) of the chelator component;
a water-soluble or water-dispersible polymer having a T_{G} greater than or equal to 20° C.; and
a water-soluble plasticizer component having a boiling point greater than 105° C. and having a formula weight of less than 5000 atomic mass units;
a solvent present in an amount less than 10 wt % of the composition, wherein the solvent is characterized by a boiling point less than or equal to 100 degrees C.;
wherein the composition is a solid at 25° C., and
wherein the composition, when mixed with deionized water at a 1:9 mass ratio, forms an aqueous mixture having a pH of about 2.5-5.5.
